(19) United States
(12) Patent Application Publication
(10) Pub. No.: US 2015/0290006 A1
(43) Pub. Date: Oct. 15, 2015

(54) METHOD OF PREVENTING OR ALLEVIATING HIGH VENOUS PRESSURE DUE TO TRICUSPID REGURGITATION IN A PATIENT

(71) Applicants: Karl STANGL, Berlin (DE); Michael LAULE, Berlin (DE)

(72) Inventors: Karl Stangl, Berlin (DE); Michael Laule, Berlin (DE)

(21) Appl. No.: 14/441,217

(22) PCT Filed: Dec. 4, 2013
(86) PCT No.: PCT/EP2013/075548

§ 371 Date: May 7, 2015

Related U.S. Application Data
(60) Provisional application No. 61/734,459, filed on Dec. 7, 2012.

(51) Int. Cl. A61F 2/94

(52) U.S. Cl. CPC A61F 2/94; A61F 2002/821

(57) ABSTRACT

The present invention is directed to a method of preventing or alleviating high venous pressure in a patient, the method comprising the steps of implanting a first biocompatible scaffold into the lumen of the vena cava inferior (VCI) of the patient, preferably at a site between the right atrium and the ostium of the hepatic veins; optionally implanting a second biocompatible scaffold into the lumen of the first biocompatible scaffold; and placement of a first valve into the lumen of the first or second biocompatible scaffold; wherein the biocompatible scaffolds and the first valve are configured and arranged to permit blood flow towards a right atrium of the patient and to prevent blood flow in an opposite direction.

In U.S. Ser. No. 10/418,663, U.S. Ser. No. 10/418,677 and U.S. Ser. No. 10/653,397, the complete content of which is incorporated herein by reference, methods and devices are disclosed for reduction of pressure effects of cardiac tricuspid valve regurgitation. In all these methods stented valves are implanted in the vena cava inferior and vena cava superior in such a way that blood flow towards the right atrium of the patient is permitted whereas blood flow into the opposite directions is prevented.

A problem with these methods is that the vena cava provides complex anatomy in the vicinity of the right atrium (RA) and a large vessel diameter in this area Thus, at present there are no stented valves available which are large enough and which can be implanted securely enough to provide reliable improvement of the situation of the patient.

The present invention provides methods which overcome at least one of the problems of the prior art.

In a first aspect, the present invention provides a method of preparation of landing zones for valve implantation into the superior caval vein and the inferior caval vein.

The present application discloses:

A method of preventing or alleviating high venous pressure in a patient, the method comprising:
implanting a first biocompatible scaffold into the lumen of the vena cava inferior (VCI) of the patient, preferably at a site between the right atrium and the ostium of the hepatic veins;
optionally implanting a second biocompatible scaffold into the lumen of the first biocompatible scaffold;
placement of a first valve into the lumen of the first or second biocompatible scaffold;
   wherein the biocompatible scaffolds and the first valve are configured and arranged to permit blood flow towards a right atrium of the patient and to prevent blood flow in an opposite direction.

A method of preventing or alleviating high venous pressure in a patient as described above, the method further comprising:
implanting a third biocompatible scaffold into the lumen of the vena cava superior (VCS) of the patient, preferably at a site between the right atrial junction and the ostium of the azygos vein;
optionally implanting a fourth biocompatible scaffold into the lumen of the first biocompatible scaffold;
placement of a second valve into the lumen of the third or fourth biocompatible scaffold;
   wherein the biocompatible scaffolds and the second valve are configured and arranged to permit blood flow towards a right atrium of the patient and to prevent blood flow in an opposite direction.

In the method of the invention, the first valve may be placed into the vena cava inferior first and thereafter the second valve may be placed into the vena cava superior; or vice versa.

In above mentioned methods, the biocompatible scaffolds and/or the valves may be implanted or placed by endolumial delivery, e.g. by delivery via a blood vessel selected from a femoral vein, a jugular vein and a subclavian vein. Endoluminal delivery may be facilitated by use of catheter-based techniques, e.g. by use of a balloon-catheter. The skilled person is well aware of means and methods suitable for endoluminal delivery of biocompatible scaffolds and/or valves.

The first, second, third and/or fourth biocompatible scaffold may be a stent or a bioadsorbable scaffold, respectively. The first, second, third and/or fourth biocompatible scaffold is preferably designed to be expandable, so that it can be implanted by introducing the scaffold in a collapsed state until the desired position is reached and by fixing the scaffold at the desired position by expanding the scaffold. The biocompatible scaffold may be self-expandable or expandable by an implantation device like e.g. an inflatable balloon. The skilled person is well aware of suitable biocompatible scaffolds, e.g. of suitable stents.

The first and second valve has at least one valve leaflet each. The at least one valve leaflet may be formed of a synthetic material or of a biologic material. Preferably, the biologic material is derived or obtained from a pericardium, e.g. from human, bovine, equine, porcine or ovine pericardium. The skilled person is well aware of valves that are suitable for use in the method of the invention. Both, venous and arterial valves are suitable. Preferably, arterial valves are used.

In the method of the invention, the first biocompatible scaffold is longer than the second biocompatible scaffold. The reduction in diameter of the vena cava inferior achieved by implantation of the first biocompatible scaffold is sufficient to allow for proper and safe placement of the first valve. In order to further improve proper and safe placement of the first valve into the relatively large lumen of the vena cava inferior, a second biocompatible scaffold may be placed into the lumen of the first biocompatible scaffold in order to further reduce the diameter of the lumen of the vena cava inferior.

In a preferred embodiment, it is provided a method of stabilization of the vessel wall and of preparing a landing zone enabling the implantation of percutaneously implantable catheter-based heart valves in the vena cava superior (VCS) and the vena cava inferior (VCI). The method comprises the implantation of a stent or a bioadsorbable scaffold into the VCS and/or the VCI enabling stabilization and fixation of a ballon expandable or selfexpanding valve and prevention of vessel rupture.

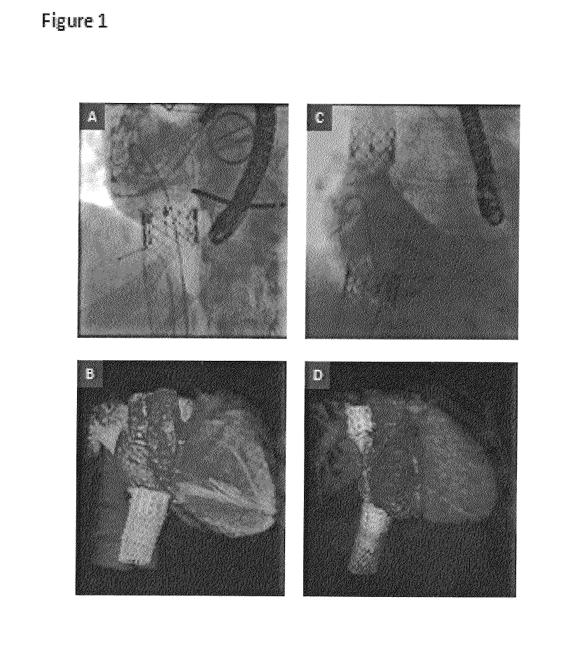

The major challenges for valve implantation in the VCI are complex anatomy, a short segment between RA and the ostium of the hepatic veins, as well as large diameter of the VCI. At present, the only suitable commercial prosthesis for this percutaneous approach is the Edwards Sapien valves. The intervention was performed as compassionate treatment. All patients provided written informed consent. The procedure was performed via the right femoral vein (20 F eSheat, Novaflex). To guarantee stable placement of the prosthesis, we prepared a landing zone by implanting a self-expanding 30/60-mm Sinus XL Stent in the VCI segment downstream of the RA. To further downsize the lumen we placed a second, shorter stent in the upper part of the first stent. The Edwards Sapien XT valve mounted on the Novaflex delivery system was then deployed (FIGS. 1A and B). In patient 3, we performed the dual valve procedure: in addition to VCI, a VCS valve was also implanted after positioning of a self-expanding stent, to reduce the risk of vessel wall damage/rupture (FIGS. 1C and D). Subsequently, the second prosthesis was implanted in the VCI by the method as described above.

In the following, the invention is further described by way of an exemplary embodiment.

Severe tricuspid regurgitation (TR) is associated with increased morbidity and mortality. In advanced TR stages, right-sided heart failure, ascites, and congestive hepatopathy increase surgical risk; alternative approaches are therefore required.

Transcatheter valve procedures are increasingly applied in clinical practice to treat aortic, mitral, and pulmonary valve diseases. Few data are available, however, on percutaneous treatment of tricuspid valve (TV). Animal experiments have demonstrated the feasibility, reduction of TR, and improvement of hemodynamics associated with percutaneous implantation of valves in central venous positions. One human case report described successful transcatheter treatment of TR with a custom-made, self-expanding heart valve for inferior vena cava implantation (VCI).

Here we describe the feasibility, technical details, as well as periprocedural and short-term outcomes of a novel first-in-man approach for implantation of the Edwards Sapien XT (approved for the aortic valve) as VCI valve: between the right atrium (RA) and the hepatic vein (i.e., single valve) and in combination with a superior vena cave (VCS) valve (dual valve). Between August and September of 2012, we treated three patients with severe symptomatic TR and contraindications to surgical repair. All patients (2 men, 1 women) had secondary TR with recurrent right heart failure despite optimal therapy (including high-dose diuretics). Due to the presence of defibrillator leads in the VCS, a single valve was implanted in the VCI in patients 1 and 2, whereas patient 3 received dual valve implantation. Echocardiography and multislice computed tomography (MSCT) were performed to assess disease severity (right heart parameters) and to evaluate carefully the relationship/distance between RA, VCI, and the hepatic veins. These investigations were repeated after one month to evaluate postinterventional results. In addition, periprocedural, in-hospital, and 30-day outcomes were assessed according to the Valve Academic Research Consortium Criteria (VARC). Procedures were performed under general anesthesia with fluoroscopic and TEE guidance.

The major challenges for valve implantation in VCI are complex anatomy, a short segment between RA and the ostium of the hepatic vein, as well as large diameter of the vena cava. At present, the only suitable commercial prosthesis for this percutaneous approach is the Edwards Sapien XT (29 mm). The intervention was performed as compassionate treatment. All patients provided written informed consent. The procedure was performed via the right femoral vein (20 F eSheat, Novaflex). To guarantee stable placement of the prosthesis, we prepared a landing zone by implanting a self-expanding 30/60-mm Sinus XL Stent in the VCI segment downstream of the RA. To further downsize the lumen we placed a second, shorter stent in the upper part of the first stent. The Edwards Sapien XT valve mounted on the Novaflex delivery system was then deployed (FIGS. 1A and B). In patient 3, we performed the dual valve procedure: in addition to VCI, a VCS prosthesis was also implanted after positioning of a self-expanding stent, to reduce the risk of vessel wall damage/rupture (FIGS. 1C and D). Subsequently, the second prosthesis was implanted in the VCI by the same technique as described above. Right ventricular angiography and echocardiography confirmed intact valve function without para-valvular leak and without regurgitation in any case. There were no periprocedural or in-hospital complications. Periinterventionally, the patients received unfractionated heparin and thereafter oral anticoagulation.

At 30 days there were no events according to VARC criteria. Valve function remained excellent throughout the follow-up period. No valve regurgitation or leak was detected. We observed free drainage of hepatic veins with only antegrade flow into the VCI. As shown in Table I, all patients improved by at least one NYHA class, and signs of right heart congestion clearly decreased. It is noteworthy that in all 3 patients RV function improved, and that RV and RA volumes as well as the diameter of the hepatic veins decreased.

In summary, percutaneous single or dual caval prosthesis implantation with the Edwards Sapien XT for severe TR is feasible and safe. This gate-keeping mechanism alleviates hepatic and peripheral congestion and leads to functional clinical amelioration. Despite our very promising short-term results, further larger controlled trials are necessary to determine the impact of this novel interventional approach on morbidity and mortality in patients with severe TR.

1. A method of preventing or alleviating high
venous pressure in a patient, the method comprising:
implanting a first biocompatible scaffold into the lumen of the vena cava inferior (VCI) the patient, preferably at a site between the right atrium and the ostium of the hepatic veins;
optionally implanting a second biocompatible scaffold into the lumen of the first biocompatible scaffold;
placement of a first valve into the lumen of the first or second biocompatible scaffold;
wherein the biocompatible scaffolds and the first valve are configured and arranged to permit blood flow towards a right atrium of the patient and to prevent blood flow in an opposite direction.

2. The method according to claim 1, further comprising:
implanting a third biocompatible scaffold into the lumen of the vena cava superior (VCS) of the patient, preferably at a site between the right atrial junction and the ostium of the azygos vein;
optionally implanting a fourth biocompatible scaffold into the lumen of the first biocompatible scaffold;
placement of a second valve into the lumen of the third or fourth biocompatible scaffold;
wherein the biocompatible scaffolds and the second valve are configured and arranged to permit blood flow towards a right atrium of the patient and to prevent blood flow in an opposite direction.

3. The method of claim 1, wherein the first valve is placed into the vena cava inferior first and thereafter the second valve is placed into the vena cava superior.

4. The method of claim 1, wherein the second valve is placed into the vena cava superior first and thereafter the first valve is placed into the vena cava inferior.

5. The method according to claim 1, wherein the biocompatible scaffolds and/or the valves are implanted or placed by endolumial delivery, e.g. by delivery via a blood vessel selected from a femoral vein, a jugular vein and a subclavian vein.

6. The method according to claim 1, wherein the first, second, third and/or fourth biocompatible scaffold is a stent or a bioadsorbable scaffold, respectively.

7. The method according to claim 1, wherein the first, second, third and/or fourth biocompatible scaffold is designed to be expandable, so that it can be implanted by introducing the scaffold in a collapsed state until the desired position is reached and by fixing the scaffold at the desired position by expanding the scaffold.

8. The method according to claim 1, wherein the first and second valve each has at least one valve leaflet formed of a synthetic material or of a biologic material.

9. The method according to claim 8, wherein the biologic material is derived or obtained from a pericardium, e.g. from human, bovine, equine, porcine or ovine pericardium.

10. The method according to claim 1, wherein the first biocompatible scaffold is longer than the second biocompatible scaffold.
